(19) United States
(12) Patent Application Publication
(10) Pub. No.: US 2015/0230730 A1
(43) Pub. Date: Aug. 20, 2015

(54) PULMONARY PLETHYSMOGRAPHY BASED ON OPTICAL SHAPE SENSING

(71) Applicant: KONINKLIJKE PHILIPS N.V., EINDHOVEN (NL)

(72) Inventors: Joerg Sabczynski, Norderstedt (DE); Bharat Ramachandran, Morganville, NJ (US)

(21) Appl. No.: 14/432,504

(22) PCT Filed: Oct. 1, 2013
(86) PCT No.: PCT/IB2013/059032

§ 371 Date: Mar. 31, 2015

Related U.S. Application Data
(60) Provisional application No. 61/714,501, filed on Oct. 16, 2012.

(51) Int. Cl. A61B 5/08; A61B 5/085; A61B 5/00; A61B 5/087

(52) U.S. Cl. CPC A61B 5/0806; A61B 5/087; A61B 5/085; A61B 5/6804; A61B 5/7278

(57) ABSTRACT

The present invention relates a pulmonary plethysmographic system (10), the system with a garment (11) wearable on the body of a mammal, e.g. a human, the garment comprising a shape sensing fiber (12) with a plurality of optical fibers (30) to facilitate optical measurements of strain along the length of the shape sensing fiber. An optical interrogation unit (13) is optically connected with the optical fibers in the shape sensing fiber for measuring the strain along the plurality of optical fibers. A processing unit (14) is processing the strain data into three-dimensional position data over time, the processing unit further being arranged for processing the position data over time into volume data indicative of pulmonary data about the mammal wearing the garment. The invention is advantageous for obtaining an improved system for pulmonary measurement providing a more realistic measurement of the pulmonary function of the mammal.

### FIELD OF THE INVENTION

The present invention relates to a pulmonary plethysmographic system based on optical shape sensing, the system may preferably be wearable. The invention also relates to a corresponding garment for enabling pulmonary plethysmography, a corresponding pulmonary plethysmographic method, and corresponding computer software for enabling a pulmonary plethysmographic method.

### BACKGROUND OF THE INVENTION

Broadly speaking, a plethysmograph may be defined as an instrument for measuring changes in volume within an organ, or whole body, typically resulting from fluctuations in the amount of blood or air it contains. Thus, it is an instrument for determining and registering variations in the size of an organ, limb, or part resulting from changes in the amount of blood or air present or passing through it.

In pulmonology, the assessment of the lung function is usually done with the help of a pulmonary plethysmograph. In short, a pulmonary plethysmograph measures the intrathoracic gas volume (TGV) and the patient's volume change during breathing. In combination with spirometry, plethysmography allows to directly measure the specific airway resistance (sRaw) as well as TGV and the airway resistance Raw. A body plethysmograph as it is used in clinical practice is a cabin like device with a volume of about 1000 liter which is almost air tight. The patient sits in the cabin and breathes through a spirometer.

Patient volume changes during breathing lead to compression and decompression of the air in the cabin. Pressure sensors measure these pressure changes, which are proportional to the patient's volume changes. Simultaneously, the airflow through the mouth of the patient is measured with the spirometer. Therefore, flow/volume diagrams can be created, which allow diagnosing various lung diseases as it will be understood by the skilled person. Instead of a cabin-based system, wearable devices have been described, which use two belts around.

U.S. Pat. No. 7,221,814, to Aston University, is an example of a wearable device. It discloses a surface profiling apparatus comprises three long period gratings (LPGs) fabricated in progressive three layered (PTL) fiber and embedded within a deformable carrier member comprising a skeleton provided between two sheets of flexible rubber skin. The LPGs are illuminated by three wavelength modulated, narrow bandwidth optical signals, each having a different wavelength and modulation frequency. A photodetector connected to three lock-in amplifiers measures the amplitudes of the first and second harmonic frequency components of the photodetector output signal corresponding to each LPG. Similar surface profiling apparatus forms the basis for respiratory function monitoring apparatus in which five LPGs are provided within each of four PTL fibers and embedded in four carrier members attached to a garment to be worn by a subject. However, the spatial resolution of this device is not believed to be sufficient for practical medical applications.

The inventors of the present invention have appreciated that an improved pulmonary plethysmographic system is of benefit, and have in consequence devised the present invention.

### SUMMARY OF THE INVENTION

It would be advantageous to achieve an improved pulmonary plethysmographic system. In general, the invention preferably seeks to mitigate, alleviate or eliminate one or more of the above-mentioned disadvantages singly or in any combination. In particular, it may be seen as an object of the present invention to provide a method that solves the above-mentioned problems, or other problems, of the prior art, in particular, but exclusively the problem of limited spatial resolution.

To better address one or more of these concerns, the invention relates in a first aspect to a pulmonary plethysmographic system,
a garment wearable on the body of a mammal, the garment comprising a shape sensing fiber with a plurality of optical fibers to facilitate optical measurements of strain along the length of the shape sensing fiber,
an optical interrogation unit, the unit being optically connected with said plurality of optical fibers in the shape sensing fiber, the optical interrogation unit being optically arranged for measuring the strain along the plurality of optical fibers, and
a processing unit, operably connected to the optical interrogation unit for processing said strain data into three-dimensional position data over time, the processing unit further being arranged for processing the position data over time into volume data indicative of pulmonary data about the mammal wearing said garment.

The invention is particularly, but not exclusively, advantageous for obtaining an improved system for pulmonary measurement of a mammal, e.g. a human patient, so that the mammal can move or exercise during the actual measurement and thereby provide a more realistic measurement of the pulmonary function of the mammal in a manner hitherto not possible or accessible within reasonable costs. In addition, the present invention provides an accurate and/or cost-effective solution because the optical based shape sensing fiber has been demonstrated by the present inventors to yield results directly applicable in clinical or medical applications.

It is worth mentioning that the up till now body plethysmographs have had several disadvantages:
Hitherto body plethysmographs cannot be used during exercise, activities of daily living, sleep, or sports.
The commonly used cabin-solution is rather expensive and consumes space.
Although the underlying physics is relatively simple, measuring subtle pressure differences in the cabin in order to calculate the patient volume is not trivial. Thus, temperature changes in the cabin due to the patient's body heat can influence the measurement. Additionally, external pressure changes may also influence the cabin pressure.

The pulmonary plethysmographic system according to the present invention disclosed in this application aims to, at least partly, solve, mitigate or overcome these problems.

It is therefore envisioned that the present invention may facilitate a much broader spectrum of use:

At the pulmonologists office:
for diagnosing lung function replacing conventional body plethysmographs
for diagnosing lung function during exercise.

At the patient's home for diagnosing lung function during activities of daily living.

In sports medicine; to monitor lung capacity and training advances of athletes.

In sleep medicine; to monitor breathing behavior during sleep.

Indications for body plethysmography may be:
for diagnosis of restrictive lung disease;
for measurement of lung volumes to distinguish between restrictive and obstructive processes;
for evaluation of obstructive lung diseases, such as bullous emphysema and cystic fibrosis, which may produce artifactually low results if measured by helium dilution or N₂ washout. With simultaneously determined volumes, an index of trapped gas (i.e., FRCplethysmograph/FRCHe dilution) can be established;
for measurement of lung volumes when multiple repeated trials are required or when the subject is unable to perform multibreath tests;
for evaluation of resistance to airflow;
for determination of the response to bronchodilators, as reflected by changes in Raw, sGaw, and VTG;
for determination of bronchial hyperreactivity in response to methacholine, histamine, or isocapnic hyperventilation as reflected by changes in VTG, Raw, and sGaw;
for following the course of disease and response to treatment, and other applications readily available to the skilled person in pulmonary science.

In the context of the present application, it is to be understood that the garment is a kind of clothing suitable for wearing by the mammal, e.g. the human, monkey, horse, dog, etc., in order to facilitate pulmonary measurements or monitoring. It is further to be understood that the garment covers at least partly the portion of the mammal's body where the measurement of the pulmonary function is relevant, i.e. the surface part(s) of the body where physical movement is taken place during respiration of the mammal.

In the context of the present application, it is to be understood that the three-dimensional position data over time may be understood as relative measurements according to the displacement of the shape sensing fiber during respiration of the mammal. By three-dimensional position data is understood sufficient data to established a position in three dimensional space, independent of the choice of coordinate system, e.g. Laplacian (XYZ), spherical, cylindrical, etc.

For the purpose of this application, the field of pulmonology may be defined as the sub-branch of medicine related to, but not limited to, the anatomy, the physiology and/or the pathology of lungs.

In one embodiment, the optical interrogation unit may be optically arranged for optical frequency domain reflectometry (OFDR) capable of performing optical shape sensing by optical measurements of strains in the shape sensing fiber. This may beneficially be performed by one or more optical fiber comprising a plurality of fiber Bragg gratings (FBG) distributed along the length of the optical fiber(s). Alternatively or additionally, this may be done by one or more optical fibers comprising a plurality of Rayleigh scattering characteristic segments distributed along the length of the optical fiber(s).

In some embodiment, the plurality of optical fibers are arranged with a central optical fiber and the other optical fibers being intertwined around said central optical fiber. In generally, N degrees of freedom in the spatial position requires N+1 optical fibers or cores in order to provide temperature compensation. Thus, for a 3 dimensional position determination, 4 optical fibers, or cores, may be provided with a central core surrounded by 3 cores intertwined around the central core. One alternative is one central core and 6 cores around the central one.

Thus, the pulmonary plethysmographic system according to the invention may have a plurality of optical fibers combined into a common optical fiber with a corresponding plurality of optical fiber cores.

Advantageously, a spatial resolution of the position data from the shape sensing fiber is below 2.5 mm, preferably below 1 mm, more preferably below 0.5 mm. It can also be even lower that this if the shape sensing fiber so facilitates. In some embodiments, the spatial resolution may also be higher, e.g. below 3, 4 or 5 mm if this is possible from a measurement point of view.

In some embodiment, it is contemplated that the shape sensing fiber may comprise further means for shape sensing, said further means being chosen from the group consisting of: electrical means, e.g. electrical gauges, mechanical means, e.g. micromechanical gauges, pneumatic means, acoustic means, and any combinations thereof, and other means readily available to the skilled person.

Beneficially, the processing unit may be arranged for transforming position data over time into surface data over time by performing an interpolation process, said surface data being subsequently transformed into volume data over time by an additional interpolation process.

In some embodiment, the optical interrogation unit and the processing unit may be integrated with, on or near the garment so as to make the system wearable by a mammal. It is contemplated that this will make pulmonary measurements possible under conditions or situation not hitherto be possible or practical. Furthermore, the garment may be suitable for providing a close-fit to the skin of the mammal wearing the garment, the shape sensing fiber being arranged relative to the garment for monitoring skin displacement. Thus, the close-fit may be provided in various ways, e.g. by stretchable, or elastic, textile, preferably at a size somewhat less than the actual size of the person to be measured upon. Preferably, there should be no folds and/or no free space between the garment and mammal to facilitate accurate pulmonary measurements.

In an embodiment, the garment may comprise a plurality of shape sensing fibers, the plurality of shape fibers being optically connected to the optical interrogation unit, and the processing unit being correspondingly adapted for processing data from a plurality of shape sensing fibers. Preferably, the one or more shape sensing fibers may be incorporated into the garment so as to cover at least part of said human's thorax and/or abdomen, preferably down to the pelvic area.

In some embodiments, the pulmonary plethysmographic system may have at least part of one, or more, shape sensing fiber(s) that forms a loop-like shape in, or on, the garment. Thus, if the fiber is rigid in its longitudinal or lengthwise direction, it may form a curved pattern in or on the garment, or textile, to provide for displacement of the fiber in its transverse direction. Thus, various patterns are envisioned for the one or more shape sensing fiber(s), e.g. a loop-like shape, partly circle shape, full circle shape, a U-shaped form, and so forth, and various combinations of shapes.

In one embodiment, optionally comprising a spirometer usable by the mammal wearing the garment, the spirometer preferably being integrated into a facemask, wherein the pulmonary data may consists of intrathoracic gas volume (TGV), specific airway resistance (sRaw), the airway resistance (Raw), diffusing capacity (DLCO), single-breath nitrogen (N₂), multiple-breath N₂ washout, pulmonary compliance, and occlusion pressure, and any combinations or equivalents thereof.

In a second aspect, the present invention relates to a garment for pulmonary plethysmography, the garment being wearable on the body of a mammal, the garment comprising a shape sensing fiber with a plurality of optical fibers to facilitate optical measurements of strain along said length,

the garment being adapted for cooperating with an associated optical interrogation unit, for being optically connected with said plurality of optical fibers in the shape sensing fiber, the optical interrogation unit being optically arranged for measuring the strain along the plurality of optical fibers, and
the garment further being adapted for cooperating with an associated a processing unit, operably connected to the optical interrogation unit for processing said strain data into three-dimensional position data over time, the processing unit further being arranged for processing the position data over time into volume data indicative of pulmonary data about the mammal wearing said garment.

In third aspect, the present invention relates to a pulmonary plethysmographic method, the method comprising

providing a garment wearable on the body of a mammal, the garment comprising a shape sensing fiber with a plurality of optical fibers to facilitate optical measurements of strain along said length,

providing an optical interrogation unit, the unit being optically connected with said plurality of optical fibers in the shape sensing fiber, the optical interrogation unit being optically arranged for measuring the strain along the plurality of optical fibers, and

providing processing unit, operably connected to the optical interrogation unit for processing said strain data into three-dimensional position data over time, the processing unit further being arranged for processing the position data over time into volume data indicative of pulmonary data about the mammal wearing said garment.

In a fourth aspect, the present invention relates to a pulmonary plethysmographic computer program product being adapted to enable a computer system comprising at least one computer having data storage means in connection therewith to implement a method according to the third aspect of the present invention.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 shows a schematic embodiment of a pulmonary plethysmographic system according to the present invention,

FIG. 2 shows a more detailed embodiment of a pulmonary plethysmographic system according to the present invention,

FIG. 3 shows a schematic perspective illustration of a shape sensing fiber with optical fiber cores according to the present invention,

FIG. 4 shows another embodiment of a pulmonary plethysmographic system according to the present invention, which is wearable by a human,

FIG. 5 shows a photograph of a garment for enabling a pulmonary plethysmographic system according to the present invention,

FIG. 6 shows a graph comparing the pulmonary plethysmographic system according to the present invention to an alternative system based on digital imaging,

FIG. 7 shows another graph comparing the pulmonary plethysmographic system according to the present invention (OSS) to an alternative system based on magnetic resonance imaging (MR), and

FIG. 8 shows a flow chart of a method according to the present invention.

### DESCRIPTION OF EMBODIMENTS

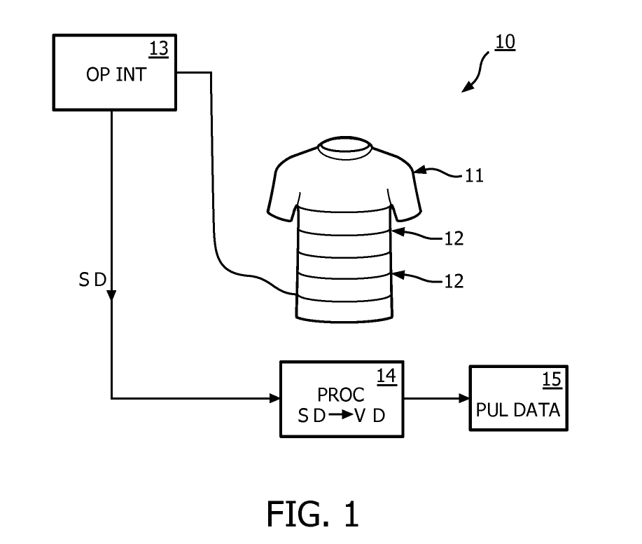

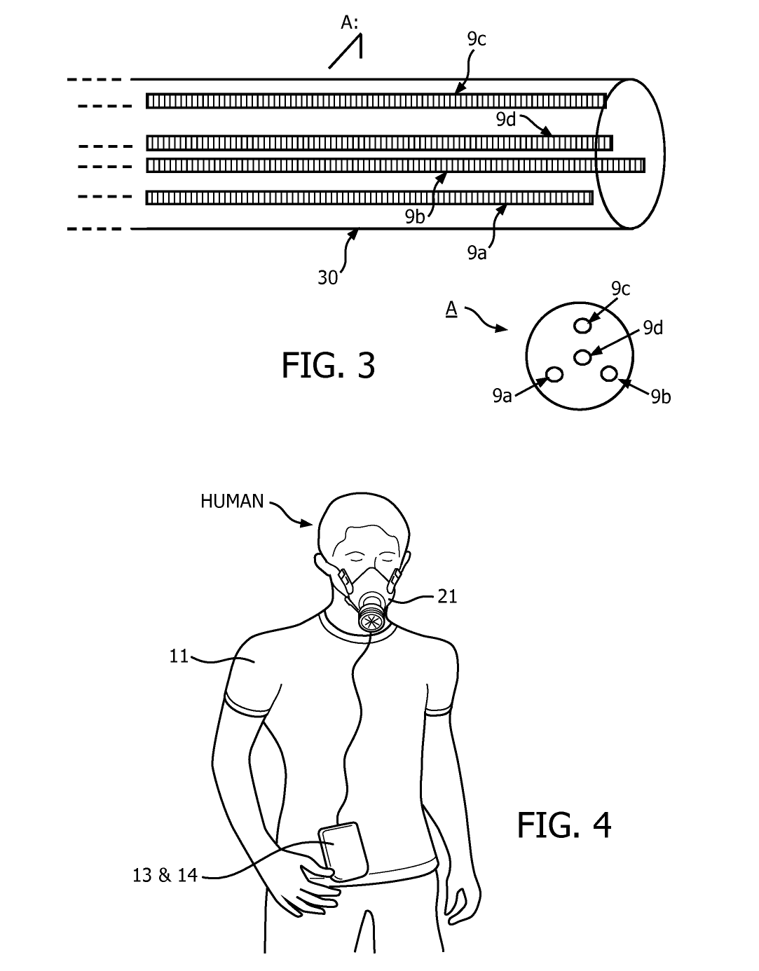

FIG. 1 shows a schematic embodiment of a pulmonary plethysmographic system 10, the system comprising a garment 11, e.g. a T-shirt-like garment, wearable on the body of a mammal (not shown), the garment comprising a shape sensing fiber 12, arranged in a peripheral pattern on the garment as shown, with a plurality of optical fibers (also not shown, but shown in FIG. 3) to facilitate optical measurements of strain along the length of the shape sensing fiber 12 on the body part where the fiber is in close contact with the body during respiration.

An optical interrogation unit 13 is also provided, the unit being optically connected with said plurality of optical fibers in the shape sensing fiber, the optical interrogation unit being optically arranged for measuring the strain along the plurality of optical fibers as will be explain in further details below.

A processing unit 14 is operably connected to the optical interrogation unit 13 for processing said strain data into three-dimensional position data over time, the processing unit further being arranged for processing the position data over time into volume data indicative of pulmonary data, PUL DATA as shown in box 15, about the mammal wearing said garment. The box 15 can be a display device of a suitable kind, or, alternatively or additionally, the pulmonary data can be stored in an electronic storage device, e.g. a hard disk.

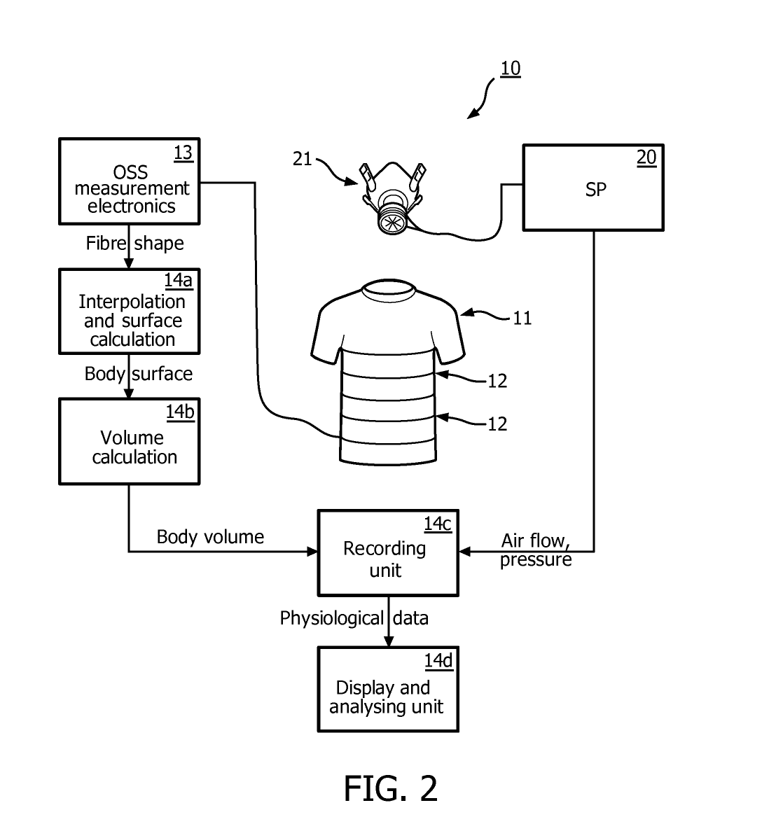

FIG. 2 shows a more detailed embodiment of a pulmonary plethysmographic system according to the present invention. The embodiment is illustrated in a schematic manner to provide an overview. In practical application, the various elements are beneficially integrated into one or more units. Thus, the surface interpolation, volume calculation, processing of flow data, storing of data, communication with other devices etc. may be implemented as software modules running on a single processor.

The wearable plethysmograph comprises of three units 13, 14, and 15 as explained above, which optionally may be integrated into the device (see FIG. 3):
1. The optical shape sensing unit with optical interrogation unit 13 for determining the volume of the patient's body
2. The spirometer unit 20 for measuring airflow during breathing
3. The processing unit 14 for recording and analyzing unit for storing measurements and displaying results PUL DATA.

With these units, the following data processing can be realized:

1. Raw data generation:
Optical shape sensing (OSS) systems are available, cf. a description of the method in patent application US 2011/0109898 entitled “Optical position and/or shape sensing”, which is hereby incorporated by reference in its entirety. These systems allow for measuring and reconstructing the exact position and shape of an optical fiber along its length. If combined with textiles, it is possible to create shape sensing garments 12, e.g. vests or shirts. This vest should be tightly fitting to the body, ensuring, that the optical fiber is in contact with the body surface. The OSS system thus measures points on the surface of the patient's body.
   2. Surface calculation:
Since there will always be a certain distance between adjacent fibers or loops of the same fiber, these surface points are only sparsely sampled. Therefore, the surface of the patient can be interpolated in box 14a.
   3. Volume calculation 14b:
The interpolated surface can be used to calculate the volume of the patient's body.
This result is then transferred to the recording unit.

Steps 1-3 above may be repeated with an update frequency of several (up to 40) Hz. Simultaneously, the spirometer unit 20 measures the airflow through the mouth and transmits it to the recording unit.

Alternatively to the processing flow described in FIG. 2, it is also be possible to record the OSS data (position data) immediately. This would direct the data i.e. from the flow chart boxes in the following alternative way: from position measurement 13, and spirometer 20 to recording at box 14c, and subsequently, possible later, to body surface calculation at box 14a and the volume calculation at box 14b, finally displayed or presenting the results at box 14d. It may also be mentioned, that is possible to have separate two recording units, one for position data (or surface or volume data) and one for airflow data from spirometer unit 20.

The body volume measurement without spirometry 20 may be sufficient for certain applications. For these applications, the spirometer may be omitted.

The simultaneously acquired volume and flow data are then transmitted to the display and analyze unit 14d for analysis by the pulmonologist, or other medical personnel. The resulting flow/volume diagrams can for example be used for diagnosis. The recording unit may also wirelessly transmit the data to a data storage unit, or to an analyzing unit in another position.

The garment or vest 11 may be additionally equipped with ECG electrodes (not shown) in order to record cardiac status simultaneously to lung function.

An accuracy achievable in-vitro with current systems is estimated to be about 2.5% for volume measurements (in infants). When the present inventors models the human body as a cylinder with height 60 cm and diameter 40 cm (30 cm) for a big (thin) person, one needs to determine the average radius of the human body with an accuracy of 2.4 mm (1.8 mm). When an accuracy of 1% is required, one needs 0.7 mm for a thin person. This is a rough estimation of the systematic error of the measurements. Statistical errors of the radius can be higher, since they will average out during integration while calculating the body volume.

This rough estimation shows, that the present invention is in a range achievable by the measurement system.

FIG. 3 shows a schematic perspective illustration of a shape sensing fiber with optical fiber cores according to the present invention. FIG. 3 shows a schematic perspective illustration of an optical fiber 10 with four optical fiber cores 9a, 9b, 9c, and 9d according to the present invention. The cores 9 have one or more FBGs along the length (the left side end portion not being shown as indicated by the broken lines). The optical cores 9 are shown to be parallel with a central axis of the optical fiber 30, but in some embodiments the optical cores may be arranged otherwise. In one preferred embodiment, the number of optical cores 9 is four and they are arranged with a central optical core 9d being parallel to the optical fiber 30 and the other three cores 9 are helically twisted around the said central core (not shown).

Using an extrinsic scattering signal by writing Bragg gratings in the cores of the optical fiber it is possible to obtain an optical shape sensing fiber. The scattering efficiency can be around 1% in magnitude. The signals of the interferometer will increase by the square root of this ratio, i.e. 10³ or 60 dB. Termination of a shape sensing fiber needs only a small amount of suppression of the end reflection, so that e.g. an 8 degree angle polished cut will suffice. All issues concerning cross talk between fiber cores, finite rejection ratio of the circulator, reflections due to multi-core connectors are mitigated. Furthermore, the lead wires will have a negligible signal with respect to the shape sensing fiber. Increasing lead wire length can easily be compensated by adding equal amount of fiber length in the reference arm of the interferometer without deterioration of the integrity of the phase measurement.

U.S. Pat. No. 7,781,724 is an example of a shape/position sensing device using fiber Bragg gratings, which is also hereby incorporated by reference in its entirety. The device comprises an optical fiber means. The optical fiber means comprises either at least two single core optical fibers or a multicore optical fiber having at least two fiber cores. In either case, the fiber cores are spaced apart such that mode coupling between the fiber cores is minimized. An array of fiber Bragg gratings (FBGs) are disposed within each fiber core and a frequency domain reflectometer is positioned in an operable relationship to the optical fiber means. In use, the device is affixed to an object. Strain on the optical fiber is measured and the strain measurements correlated to local bend measurements. Local bend measurements are integrated to determine position and/or shape of the object. An inherent disadvantage is that for typical FBG configurations, the detector of the reflectometer must have a relatively large dynamic range to encompass the information in the ‘wings’ of the spectral band.

Alternatively, or additionally, to fiber Bragg gratings (FBG) one can apply Rayleigh scattering to measure 3 dimensional position data in the context of the present invention.

One principle exploited for OSS is distributed strain measurement in optical fiber with characteristic Rayleigh scatter patterns.

Rayleigh scatter occurs as a result of random fluctuations of the index of refraction in the fiber core, inherent to the fiber manufacturing process. These random fluctuations can also be modeled as a Bragg grating with a random variation of amplitude and phase along the grating length. If strain or temperature change is applied to the optical fiber, the characteristic Rayleigh scatter pattern changes. An optical measurement can be performed first with no strain/temperature stimulus applied to the fiber to produce a reference scatter pattern and then again after induction of strain/temperature. Cross-correlation of the Rayleigh scatter spectra of the fiber in the strained/unstrained states determines the spectral shift resulting from the applied strain. This wavelength Δλ or frequency shift Δv of the backscattered pattern due to temperature change ΔT or strain along the fiber axis E is very similar to the response of a fiber Bragg grating:

$$\frac{Δλ}{λ} = - \frac{Δ v}{v} = {K}_{T} Δ T + {K}_{ɛ} ɛ , \qquad ( 11 )$$

where the temperature coefficient K_{T} is the sum of the thermal expansion and thermo-optic coefficient. The strain coefficient K_{ε} is a function of group index n, the components of the strain optic tensor p_{i,j} and Poisson's ratio

$$v \text{:} {K}_{ɛ} = 1 - \frac{{n}_{eff}^{2}}{2} ( {p}_{12} - v ( {p}_{11} + {p}_{12} ) ) .$$

Thus a shift in temperature or strain is merely a linear scaling of the spectral wavelength shift Δλ.

Optical Frequency Domain Reflectometry (OFDR) essentially performs frequency encoding of spatial locations along the fiber which enables distributed sensing of local Rayleigh reflection patterns.

In OFDR, the laser wavelength or optical frequency is linearly modulated over time. For coherent detection, the backscattered wave is mixed with a coherence reference wave at the detector. The detector receives a modulated signal owing to the change of constructive to destructive interference and vice versa while scanning the wavelength. Its frequency Ω marks the position s on the fiber and its amplitude is proportional to the local backscattering factor and the total amplitude attenuation factor of forward plus backward propagation through the distance s. By performing a Fourier transform of the detector signal using, for example, a spectrum analyzer, this method allows for simultaneous recovery of the backscattered waves from all points s along the fiber. Thus, strain on different portions of the fiber can be determined by measuring spectral shifts of the characteristic Rayleigh scattering pattern using any number of shift-detection or pattern-matching methods (e.g. block-matching with cross-correlation or other similarity metric, computation of signal phase change, etc.) in combination with OFDR.

A shape sensing device can be built using the above distributed strain measurement methodology when either 2 or more optical fibers are in a known spatial relationship such as when integrated in a multi-core shape sensing fiber. Based on a reference shape or location with reference Rayleigh scatter patterns (or reference strains) new shapes can be reconstructed using relative strains between fibers in a known/given/fixed spatial relationship.

FIG. 4 shows another embodiment of a pulmonary plethysmographic system according to the present invention, which is wearable by a human,

The main element of the invention is a vest like garment, which is made of elastic fabric and which is tightly fitting to the patient (see FIG. 4). Interwoven into the vest are fibers of a fiber-optic shape sensing system. Such a system allows identifying the position and shape of thin optical fibers with high spatial and time resolution.

The vest covers the patient's thorax as well as the abdomen down to the pelvic area. Therefore, it is possible to measure the shape of the patient's body and calculate its volume. The optical fibers are connected to the measurement electronics which can be attached to or integrated into the vest 11.

In addition, the patient may wear a facemask 21 with integrated spirometer (and mouth pressure sensors or shutters, if clinically relevant). The airflow (and pressure) as measured by the spirometer 21 is recorded together with the shape and volume of the patient's body. The spirometer may optionally also be equipped with sensors to analyze expiration gas of the mammal.

The spirometer unit may be further equipped with shutters to measure the mouth pressure during respiratory effort against closed shutter. Together with the measured volume changes, this type of measurement allows to calculate thoracic gas volume (TGV), a number necessary to calculate airway resistance Raw from the specific airway resistance sRaw.

It is important to realize, that the shape sensing technology integrated into the vest does not suffer from drawbacks of other technologies, which use belt-like devices to estimate chest or abdomen circumference. These belts might shift during movements and thus cannot be used to reliably measure the body volume neither during exercise nor during sleep. So called “fiber optic respiratory plethysmography” belongs into this category since it measures the circumference of the abdomen or thorax with a belt-like device only. The shape sensing vest device allows replacing a cabin-based body plethysmograph with a much smaller device. In addition, it is portable and thus allows to measure lung function during exercise or activities of daily living similar to an ambulatory electrocardiography device.

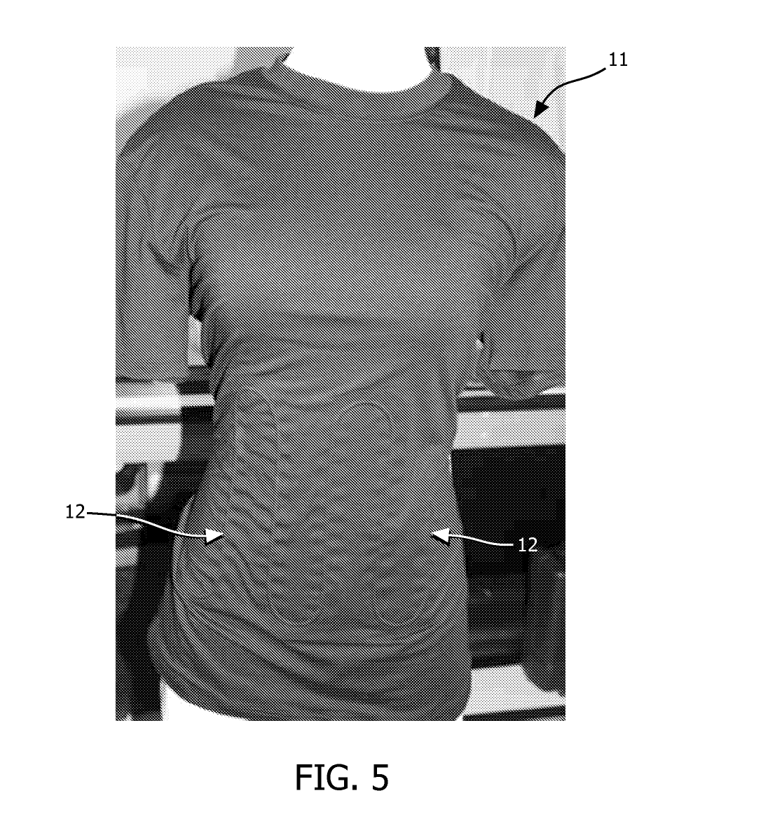

FIG. 5 shows a photograph of a garment 11 for enabling a pulmonary plethysmographic system according to the present invention.

The pulmonary plethysmographic system may have at least part of the shape sensing fiber interwoven into the garment 11.

Alternatively, at least part of the shape sensing fiber is confined by one or more compartment in, or on, the garment 11. It can also be integrated with the garment in a layer by layer manner. Possibly, the shape sensing fiber may be confined in a tube in the garment 11.

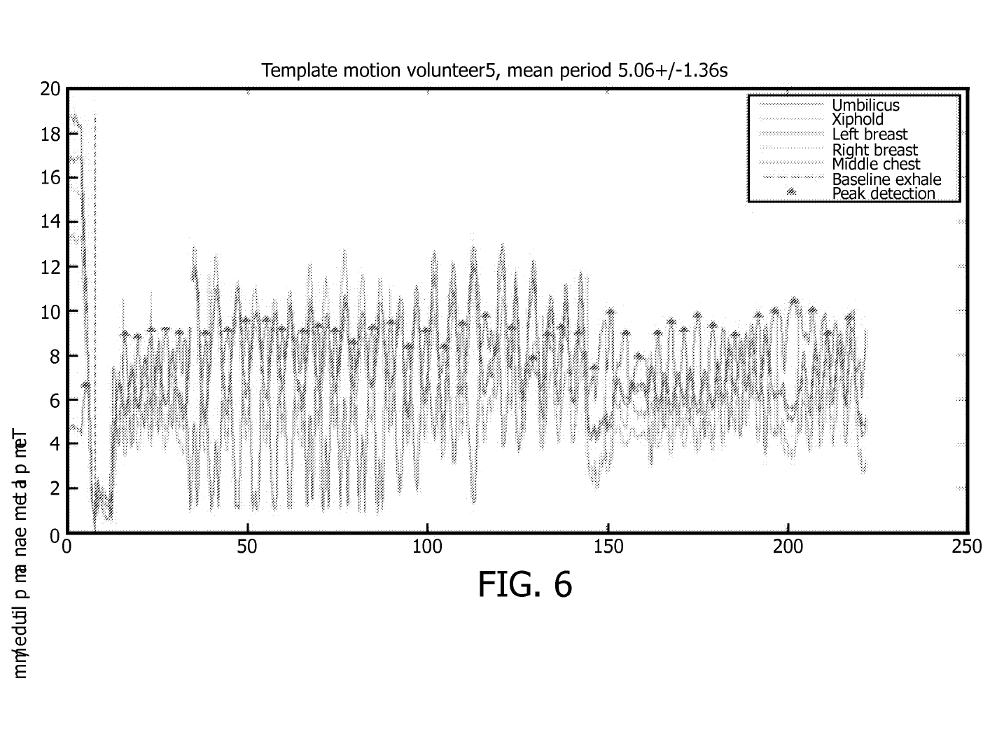

FIG. 6 shows a graph comparing the pulmonary plethysmographic system according to the present invention to an alternative system based on digital imaging. The abdomen and chest curve which is generated by the present invention shows a good correlation with the digital imaging data, thereby validating the pulmonary plethysmographic system according to the present invention.

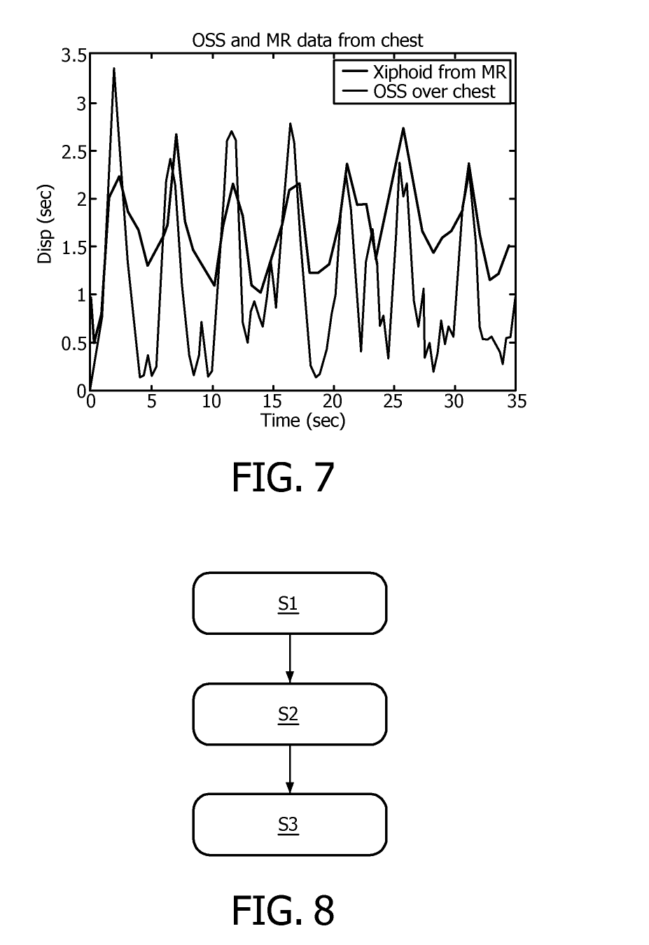

FIG. 7 shows a graph comparing the pulmonary plethysmographic system according to the present invention (OSS) to an alternative system based on magnetic resonance imaging (MR). Again, the OSS curve generated by present invention shows a good correlation with the chest curve obtained by MR thereby further validating the pulmonary plethysmographic system according to the present invention.

FIG. 8 shows a flow chart of a method according to the present invention, the method comprising:

S1 providing a garment 12 wearable on the body of a mammal, the garment comprising a shape sensing fiber with a plurality of optical fibers to facilitate optical measurements of strain along said length,
S2 providing an optical interrogation unit, the unit being optically connected with said plurality of optical fibers in the shape sensing fiber, the optical interrogation unit being optically arranged for measuring the strain along the plurality of optical fibers, and
S3 providing processing unit, operably connected to the optical interrogation unit for processing said strain data into three-dimensional position data over time, the processing unit further being arranged for processing the position data over time into volume data indicative of pulmonary data about the mammal wearing said garment.

In short, the present invention relates a pulmonary plethysmographic system 10, the system with a garment 11 wearable on the body of a mammal, e.g. a human, the garment comprising a shape sensing fiber 12 with a plurality of optical fibers 30 to facilitate optical measurements of strain along the length of the shape sensing fiber. An optical interrogation unit 13 is optically connected with the optical fibers in the shape sensing fiber for measuring the strain along the plurality of optical fibers. A processing unit 14 is processing the strain data into three-dimensional position data over time, the processing unit further being arranged for processing the position data over time into volume data indicative of pulmonary data about the mammal wearing the garment. The invention is advantageous for obtaining an improved system for pulmonary measurement providing a more realistic measurement of the pulmonary function of the mammal.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality.

In the claims, or the description, the mentioning of “at least one of a first entity, a second entity, and third entity” does not necessarily mean that each of the first entity, the second entity, and third entity are present, hence only the second entity may be present, or alternatively, only the first entity and third entity may be present, and so forth with more entities. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A pulmonary plethysmographic system, the system comprising
a garment wearable on the body of a mammal, the garment comprising a shape sensing fiber with a plurality of optical fibers to facilitate optical measurements of strain along the length of the shape sensing fiber,
an optical interrogation unit, the unit being optically connected with said plurality of optical fibers in the shape sensing fiber, the optical interrogation unit being optically arranged for measuring the strain along the plurality of optical fibers, and
a processing unit, operably connected to the optical interrogation unit for processing said strain data into three-dimensional position data over time, the processing unit further being arranged for processing the position data over time into volume data indicative of pulmonary data about the mammal wearing said garment.

2. The pulmonary plethysmographic system according to claim 1, wherein the optical interrogation unit is optically arranged for optical frequency domain reflectometry (OFDR) capable of performing optical shape sensing by optical measurements of strains in the shape sensing fiber.

3. The pulmonary plethysmographic system according to claim 1, wherein one or more optical fiber comprising a plurality of fiber Bragg gratings (FBG) distributed along the length of the optical fiber(s).

4. The pulmonary plethysmographic system according to claim 1, wherein one or more optical fibers comprising a plurality of Rayleigh scattering characteristic segments distributed along the length of the optical fiber(s).

5. The pulmonary plethysmographic system according to claim 1, wherein the plurality of optical fibers are arranged with a central optical fiber and the other optical fibers being intertwined around said central optical fiber.

6. The pulmonary plethysmographic system according to claim 1, wherein the plurality of optical fibers is combined into a common optical fiber with a corresponding plurality of optical fiber cores.

7. The pulmonary plethysmographic system according to claim 1, wherein a spatial resolution of the position data from the shape sensing fiber below 2.5 mm, preferably below 1 mm, more preferably below 0.5 mm.

8. The pulmonary plethysmographic system according to claim 1, wherein the shape sensing fiber comprises further means for shape sensing, said further means being chosen from the group consisting of: electrical means, mechanical means, pneumatic means, acoustic means, and any combinations thereof.

9. The pulmonary plethysmographic system according to claim 1, wherein the processing unit is arranged for transforming position data over time into surface data over time by performing an interpolation process, said surface data being subsequently transformed into volume data over time by an additional interpolation process.

10. The pulmonary plethysmographic system according to claim 1, wherein the optical interrogation unit and the processing unit are integrated with, on or near the garment so as to make the system wearable by a mammal.

11. (canceled)

12. (canceled)

13. The pulmonary plethysmographic system according to claim 1, wherein the one or more shape sensing fibers is incorporated into the garment so as to cover at least part of said human's thorax and/or abdomen, preferably down to the pelvic area.

14. The pulmonary plethysmographic system according to claim 1, optionally comprising a spirometer usable by the mammal wearing the garment, the spirometer preferably being integrated into a facemask, wherein the pulmonary data consists of intrathoracic gas volume, specific airway resistance, the airway resistance, diffusing capacity, single-breath nitrogen, multiple-breath N₂ washout, pulmonary compliance, and occlusion pressure, and any combinations or equivalents thereof.

15. A garment for pulmonary plethysmography, the garment being wearable on the body of a mammal, the garment comprising a shape sensing fiber with a plurality of optical fibers to facilitate optical measurements of strain along said length,
the garment being adapted for cooperating with an associated optical interrogation unit, for being optically connected with said plurality of optical fibers in the shape sensing fiber, the optical interrogation unit being optically arranged for measuring the strain along the plurality of optical fibers, and
the garment further being adapted for cooperating with an associated a processing unit, operably connected to the optical interrogation unit for processing said strain data into three-dimensional position data over time, the processing unit further being arranged for processing the position data over time into volume data indicative of pulmonary data about the mammal wearing said garment.

16. A pulmonary plethysmographic method, the method comprising
providing a garment wearable on the body of a mammal, the garment comprising a shape sensing fiber with a plurality of optical fibers to facilitate optical measurements of strain along said length,
providing an optical interrogation unit, the unit being optically connected with said plurality of optical fibers in the shape sensing fiber, the optical interrogation unit being optically arranged for measuring the strain along the plurality of optical fibers, and
providing processing unit, operably connected to the optical interrogation unit for processing said strain data into three-dimensional position data over time, the processing unit further being arranged for processing the position data over time into volume data indicative of pulmonary data about the mammal wearing said garment.

17. A pulmonary plethysmographic computer program product being adapted to enable a computer system comprising at least one computer having data storage means in connection therewith to implement a method according to claim 16.
